(19) United States
(12) Patent Application Publication
(10) Pub. No.: US 2026/0341439 A1
(43) Pub. Date: Oct. 29, 2026

(54) INTEGRATED SYSTEM FOR MEDICAL INFORMATION MANAGEMENT AND RESOURCE OPTIMIZATION

(71) Applicant: Grethel Bettina Racines Abrego, Miami, FL (US)

(72) Inventor: Grethel Bettina Racines Abrego, Miami, FL (US)

(21) Appl. No.: 19/466,104

(22) Filed: Jan. 30, 2026

(51) Int. Cl. G16H 10/65; G16H 20/00; G16H 40/20

(52) U.S. Cl. CPC G16H 10/65; G16H 20/00; G16H 40/20

(57) ABSTRACT

A system is disclosed for integrated medical information management and healthcare resource optimization. The system includes a patient associated device configured to store essential clinical information, a medical interface configured to retrieve such information at the point of care, and a computational processing module configured to integrate and analyze the retrieved data in accordance with stored clinical protocols. The system provides real time clinical decision support, reduces manual data entry requirements, and improves operational efficiency in time critical healthcare environments.

### CROSS REFERENCE TO RELATED APPLICATIONS

This application claims the benefit of U.S. Provisional Patent Application No. 63/812,024, filed May 26, 2025, the entire contents of which are hereby incorporated by reference.

### BACKGROUND OF THE INVENTION

The present invention relates to health information systems and patient flow management. Contemporary healthcare environments rely on fragmented electronic health record platforms, causing critical patient information to remain distributed across isolated data silos. Such fragmentation limits immediate access to essential clinical data at the point of care.

In high pressure environments such as Emergency Departments, delays in accessing accurate clinical information contribute to increased clinical risk, operational saturation, and inefficiencies in patient triage and treatment authorization.

Accordingly, there exists an unresolved technical need for an integrated system capable of automatically retrieving patient data, unifying information across heterogeneous clinical sources, and assisting medical personnel through computational processing to reduce delays in care delivery and minimize human error.

### SUMMARY OF THE INVENTION

The present invention provides an integrated system for medical information management and healthcare resource optimization. The system comprises a patient associated device configured to store or transmit clinical information, a reader device configured to obtain and transfer such information, and a computational processing module configured to integrate and analyze the information.

The integration of these components forms a hardware and software ecosystem designed to enable immediate retrieval of clinical information, incorporation of newly generated data, and support of clinical decision making. The system improves continuity of care across healthcare environments while reducing operational latency and administrative burden.

### BRIEF DESCRIPTION OF THE DRAWINGS

FIG. 1, illustrates a flowchart showing an example operational sequence of the integrated system, including interaction with a patient associated device, data acquisition via a reader device, computational analysis, optional laboratory testing, medical authorization, and treatment execution.

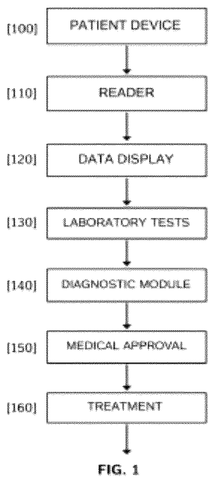

### DETAILED DESCRIPTION OF THE INVENTION

According to various embodiments of the present invention, an open and scalable system architecture is provided for the management and integration of medical information across healthcare environments.

In one embodiment, the patient associated device comprises a passive or active data carrier, such as a wearable device, a microchip card, or an implantable device. The patient associated device is configured to securely store unique health identifiers, allergy information, chronic conditions, current medications, and other clinically relevant data.

The system further includes reader devices positioned at points of triage, admission, or treatment. The reader devices are configured to communicate with the patient associated device using like optical scanning technologies or other available, to automate patient identification and data retrieval.

The computational processing system is configured to receive information from the reader devices and to integrate such information with additional clinical and operational data. In certain implementations, the computational processing system executes machine learning algorithms to analyze the integrated information and generate decision support outputs for medical personnel.

In optional embodiments, the system may interface with external diagnostic or therapeutic subsystems, including laboratory testing systems, treatment authorization systems, or medication delivery systems, wherein execution of any clinical action requires prior validation by authorized medical personnel.

What is claimed is:

1. A system comprising: a patient associated device configured to store and provide access to medical information of an individual, wherein, when the individual does not already possess the patient associated device, the device can be assignable by a healthcare facility for use during a medical encounter and for subsequent encounters with the aim of maintaining access to patient information and monitoring information; at least one medical interface system configured to retrieve the medical information from the patient associated device and to capture additional clinical data generated during a healthcare encounter; and a computational processing system in communication with the medical interface system, the computational processing system configured to receive and integrate the medical information from the patient associated device with the additional clinical data, analyze the integrated information by comparison with clinical protocols stored in at least one database, and generate, in real time, a clinical decision support output configured to reduce latency in delivery of medical care.

2. The system of claim 1, wherein the patient associated device is selected from the group consisting of a handheld device, a wearable device, an implantable device, and combinations thereof.

3. The system of claim 1, wherein the medical information comprises at least one of: identity identifiers, chronic condition records, medical records, and any historical physiological parameters.

4. The system of claim 1, wherein the computational processing system further comprises an artificial intelligence module configured to predict clinical risk levels based on integration of the medical information of patients and operational load data of a healthcare environment.

5. The system of claim 1, wherein the medical interface system comprises a scanner integrated into a hospital admission or triage station and configured to automate patient registration without manual data entry.

6. The system of claim 1, wherein the computational processing system is further configured to transmit treatment administration instructions to an external therapeutic delivery system only after prior validation by authorized medical personnel.

7. The system of claim 1, wherein the computational processing system is configured to communicate with external diagnostic or therapeutic subsystems for executing medically authorized actions.
